(19) United States
(12) Patent Application Publication
(10) Pub. No.: US 2009/0005686 A1
(43) Pub. Date: Jan. 1, 2009

(54) ULTRASONIC DIAGNOSTIC APPARATUS

(76) Inventors: Koji Yanagihara, Tokyo (JP); Kiyoshi Matsumura, Tokyo (JP); Osamu Furuta, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/200,869

(22) Filed: Aug. 28, 2008

(30) Foreign Application Priority Data
Mar. 9, 2007 (JP) 2007-227701

(51) Int. Cl. A61B 8/14

(52) U.S. Cl. 600/459

(57) ABSTRACT

An ultrasonic diagnostic apparatus includes: an operating device for an operator to input an instruction; a probe for transmitting and receiving ultrasonic waves; an image display device for displaying an ultrasonic image; and an processing device which drives the probe based on the instruction, produces the ultrasonic image, and displays the image on the image display device. The processing device is mounted on an installation platform or placed on a floor. A cable receiving space is provided inside the processing device or the installation platform for receiving a cable of probe. A probe connector is provided above an opening of the cable receiving space such that the cable is received into the cable receiving space in the shape of a U. A movable pulley is hung from a lower end of the U-shaped cable, and the movable pulley is supported on a rail in a vertically movable manner.

### CROSS REFERENCE TO RELATED APPLICATIONS

This application claims the benefit of Japanese Patent Application No. 2007-227701 filed Sep. 3, 2007, and hereby incorporated by reference in its entirety.

### BACKGROUND OF THE INVENTION

The subject matter disclosed herein relates to an ultrasonic diagnostic apparatus, and in particular, relates to an ultrasonic diagnosis apparatus that can prevent a cable of a probe from contacting and giving a patient discomfort.

A conventional ultrasonic diagnostic apparatus includes: an operating device for an operator to input an instruction; a probe for transmitting and receiving ultrasonic waves; an image display device for displaying an ultrasonic image; and a processing device which controls driving the probe, producing the ultrasonic image based on a received signal, and displaying it. The operating device, probe, image display device, and processing device can move as one unit. The cable of the probe is pulled out of a probe cable receiving device under the operating device (for example, see Japanese Unexamined Patent Publication No. 2004-49588.)

In the conventional ultrasonic diagnostic apparatus, in a case where an operator can perform operations while sitting, a height of an upper surface of the operating device is about 100 cm or less. Therefore, a height at which the cable of the probe is pulled out of the probe cable receiving device is about 95 cm or less.

On the other hand, it is possible that a height of a bed on which a patient lies is, for example, 60 cm and a body of the patient is, for example, 25 cm thick. Therefore, it is possible that a distance from a floor to a body surface of the patient is 85 cm.

Then, even if the height at which the cable of the probe is pulled out of the probe cable receiving device is 95 cm at the maximum, the remaining distance to the body surface of the patient is only 10 cm. As a result, when the cable gets loose, it may contact and give the patient discomfort.

### BRIEF DESCRIPTION OF THE INVENTION

It is desirable that the problem described previously is solved.

In a first aspect of the invention, there is provided an ultrasonic diagnostic apparatus, including: an operating device (1) for an operator to input an instruction; a probe (2) for transmitting and receiving ultrasonic waves; an image display device (3) for displaying an ultrasonic image; and an processing device (4) which controls driving the probe (1) based on the instruction, producing the ultrasonic image based on a received signal, and displaying it on the image display device (3), wherein the operating device (1) is formed separately from the processing device (4); wherein the processing device (4) is mounted on an installation platform (5) or placed on a floor (F); wherein a cable receiving space (5a) is provided inside the processing device (4) or the installation platform (5) for receiving a cable (6) of the probe (2); wherein an opening (5b) is provided at a position equal to or higher than 120 cm from the floor (F); and wherein the cable (6) of the probe (2) is received in the cable receiving space (5a) through the opening (5b).

According to the ultrasonic diagnostic apparatus of the first aspect, the operating device (1) and the processing device (4) are formed separately. Therefore, it is possible to mount the processing device (4) on the mounting platform (5) or raise the height of the processing device (4) without being influenced by the height of the operating device (1). As a result, it becomes possible to provide the cable receiving space (5a) inside the processing device (4) or mounting platform (5) and to provide the opening (5b) of the cable receiving space (5a) at the position equal to or higher than 120 cm from the floor (F). Therefore, the cable (6) comes in or goes out at a position sufficiently higher than that of a patient laying on a bed (B), preventing the cable (6) from contacting and giving the patient discomfort.

In a second aspect of the invention, there is provided an ultrasonic diagnostic apparatus according to the first aspect, wherein a probe connector (9) is provided above the opening (5b) of the processing device (4).

According to the ultrasonic diagnostic apparatus of the second aspect, the probe connector (9) is provided above the opening (5b). Therefore, the cable connector (6a) of the cable (6) can be attached to and detached from the probe connector (9) of the processing device (4) without being disturbed by the cable (6).

In a third aspect of the invention, there is provided an ultrasonic diagnostic apparatus according to the second aspect, wherein the cable (6) is bent in a U-shaped manner from the opening (5b) and is received in the cable receiving space (5a).

According to the ultrasonic diagnostic apparatus of the third aspect, only one portion at a lower end of the U-shaped cable (6) is bent with a small curvature, preventing the cable (6) from being damaged.

In a fourth aspect of the invention, there is provided an ultrasonic diagnostic apparatus according to the third aspect, wherein a movable pulley (10) is hung from a lower end of the U-shaped cable (6).

According to the ultrasonic diagnostic apparatus of the fourth aspect, the cable (6) is prevented from being bent with a curvature smaller than a radius (for example, 5 mm or over) of the movable pulley (10).

In a filth aspect of the invention, there is provided an ultrasonic diagnostic apparatus according to the fourth aspect, wherein the movable pulley (10) is supported on a rail (12) in a vertically movable manner.

According to the ultrasonic diagnostic apparatus of the fifth aspect, the movable pulley (10) moves vertically while being supported on the rail (12), which improves the stability and reliability.

In a sixth aspect of the invention, there is provided an ultrasonic diagnostic apparatus, including: an operating device (1) for an operator to input an instruction; a probe (2) for transmitting and receiving ultrasonic waves; an image display device (3) for displaying an ultrasonic image; and an processing device (4) which controls driving the probe (1) based on the instruction, producing the ultrasonic image based on a received signal, and displaying it on the image display device (3), wherein the processing device (4) is mounted on an installation platform (5) or placed on a floor (F); wherein a cable receiving space (5a) is provided inside the processing device (4) or the installation platform (5) for receiving a cable (6) of the probe (2); wherein a probe connector (9) is provided above an opening (5b) of the cable receiving space (5a); wherein the cable (6) of the probe (2) is received into the cable receiving space (5a) in the shape of U from the opening (5b); wherein a movable pulley (10) is hung from a lower end of the U-shaped cable (6); and wherein the movable pulley (10) is supported on a rail (12) in a vertically movable manner.

According to the ultrasonic diagnostic apparatus of the sixth aspect, the operation device (1) and the processing device (4) are formed separately. Therefore, it is possible to mount the processing device (4) on the mounting platform (5) or to raise the height of the processing device (4) without being influenced by the height of the operating device (1). As a result, it becomes possible to provide the cable receiving space (5a) inside the processing device (4) or the mounting platform (5). Therefore, it becomes possible for the cable (6) to come in and go out at a position sufficiently higher than that of a patient lying on a bed (B), preventing the cable (6) from contacting and giving the patient discomfort. Also, the probe connector (9) is provided at the position above the opening (5b). Therefore, a cable connector (6a) of the cable (6) can be attached to or detached from the probe connector (9) of processing device (4) without being disturbed by the cable (6). Moreover, only one portion at the lower end of the U-shaped cable is bent with a small curvature, preventing the cable (6) from being damaged. Moreover, it is possible to prevent the cable (6) from bending with a curvature smaller than a radius (for example, 5 mm or over) of the movable pulley (10).

Furthermore, since the movable pulley (10) moves vertically while being supported on the rail (12), its stability and reliability can be improved.

In a seventh aspect of the invention, there is provided an ultrasonic diagnostic apparatus according to any one of the fourth to sixth aspects, wherein the movable pulley (10) is urged downwards by a weight (11).

According to the ultrasonic diagnostic apparatus of the seventh aspect, the weight (11) provides a force to pull the cable (6) into the cable receiving space (5a) smoothly.

In an eighth aspect of the invention, there is provided an ultrasonic diagnostic apparatus according to any one of the fourth to seventh aspects, wherein a cable lock (13) is provided to prevent the disconnection of the cable (6) from the movable pulley (10).

According to the ultrasonic diagnostic apparatus of the eighth aspect, the disconnection of the cable (6) from the movable pulley (10) is prevented, improving the stability and reliability.

In a ninth aspect of the invention, there is provided an ultrasonic diagnostic apparatus according to the eighth aspect, wherein the cable lock (13) is comprised of a rotatable lever-like member.

According to the ultrasonic diagnostic apparatus of the ninth aspect, the disconnection of the cable (6) from the movable pulley (10) is prevented by a simple structure.

In a tenth aspect of the invention, there is provided an ultrasonic diagnostic apparatus according to the eighth or ninth aspect, wherein a cover (14) is provided over a portion of the processing device (4) or the mounting platform (5) 120 cm or less from the floor (F) which slides vertically to allows the access to the inside.

According to the ultrasonic diagnostic apparatus of the tenth aspect, the cover (14) is vertically slid to allow the access to an inner portion of the processing device (4) or the mounting device (5) 120 cm or less from the floor (F), making the maintenance work easier.

In an eleventh aspect of the invention, there is provided an ultrasonic diagnostic apparatus according to the tenth aspect, wherein the cable (6) can be attached to or detached from the movable pulley (10) when the cable lock (13) is released.

According to the ultrasonic diagnostic apparatus of the eleventh aspect, when removing a probe (2) not to be used from the processing device (4), it becomes possible to remove the cable (6) from the movable pulley (10) and, also, when attaching a probe (2) to be used to the processing device (4), it becomes possible to mount the cable (6) on the movable pulley (10).

In a twelfth aspect of the invention, there is provided an ultrasonic diagnostic apparatus according to the tenth or eleventh aspect, wherein a movable-pulley lock (15) is provided for holding the movable pulley (10) at a position of the rail (12) equal to or higher than 60 cm from the floor.

According to the ultrasonic diagnostic apparatus of the twelfth aspect, the movable pulley (10) from which the cable (6) is removed can be held at the position equal to or higher than 60 cm from the floor, making it easy to mount a cable (6) of a probe (2) to be used next for the movable pulley (10).

In a thirteenth aspect of the invention, there is provided an ultrasonic diagnostic apparatus according to the twelfth aspect, wherein the movable-pulley lock (15) is comprised of a seesaw-like member that varies its position in a seesaw-like manner.

According to the ultrasonic diagnostic apparatus of the thirteenth aspect, the movable pulley (10) can be prevented from descending by a simple structure.

In a fourteenth aspect of the invention, there is provided an ultrasonic diagnostic apparatus according to any one of the first to thirteenth aspects, wherein a cable stopper (7) is provided for preventing the cable (6) from coming into and going out of the cable receiving space (5a).

According to the ultrasonic diagnostic apparatus of the fourteen aspect, the cable (6) is prevented from going out of the opening (5b) or being pulled into the cable receiving space (5a) against the operator's will.

In a fifteenth aspect of the invention, there is provided an ultrasonic diagnostic apparatus according to the fourteenth aspect, wherein the cable stopper (7) is a slit (7a) which is narrower than an the outside diameter of the cable (6).

According to the ultrasonic diagnostic apparatus of the fifteenth aspect, the cable (6) is inserted in the slit (7a) so that the cable (6) is stopped in an engaged state by the elasticity of the cable (6), and the structure can also be simple.

In a sixteenth aspect of the invention, there is provided an ultrasonic diagnostic apparatus according to any one of the first to fifteenth aspects, wherein there is provided, in the opening (5b), a roller (8) which may hit an undersurface of the cable (6).

According to the ultrasonic diagnostic apparatus of the sixteenth aspect, the cable (6) comes into and goes out of the opening (5b) smoothly because of the roller (8).

In a seventeenth aspect of the invention, there is provided an ultrasonic diagnostic apparatus according to any one of the first to sixteenth aspects, wherein the thickness of a portion of the processing device (4) or the mounting platform (5) equal to or lower than 70 cm from the floor (F) is 10 cm or less.

According to the ultrasonic diagnostic apparatus of the seventeenth aspect, the processing device (4) is mounted on the mounting platform (5) whose portion equal to or lower than 70 cm from the floor (F) is 10 cm thick or less. Also, when the processing device (4) is placed on the floor (F), the thickness of the portion of the processing device (4) equal to or lower than 70 cm from the floor (F) is 10 cm or less. As a result, suppose a spacing between the bed (B) and the wall (W) is 11 cm, for example, the processing device (4) can be installed along the wall (W). Further, a height of an ordinary bed (B) is less than 70 cm. As a result of the above, the space for the operator becomes larger than that of the conventional case, making it possible to effectively utilize the space of the room in which the ultrasonic diagnostic apparatus is installed.

In all eighteenth aspect of the invention, there is provided an ultrasonic diagnostic apparatus according to any one of the first to seventeenth aspects, wherein a back surface of the processing device (4) or the mounting platform (5) is not uneven or a level of unevenness is 1 cm or less.

According to the ultrasonic diagnostic apparatus of the eighteenth aspect, the back surface of the processing device (4) or the mounting platform (5) is substantially flat. Therefore, with almost no spacing provided, the processing device (4) can be installed along the wall (W).

In a nineteenth aspect of the invention, there is provided an ultrasonic diagnostic apparatus according to any one of the first to eighteenth aspects, wherein the processing device (4) or the mounting platform (5) has a lip-resistant leg (16).

According to the ultrasonic diagnostic apparatus of the nineteenth aspect, even if a length of a contact area in the thickness direction of the processing device (4) or the mounting platform (5) is 10 cm or less, the processing device (4) or the mounting platform (5) is prevented from tipping over by the tip-resistant leg (16).

In a twentieth aspect of the invention, there is provided an ultrasonic diagnostic apparatus according to any one of the first to nineteenth aspects, wherein the image display device (3) is supported by an arm (17) extending from a column support (19) provided along the processing device (4) or an arm (17) extending from the processing device (4).

According to the ultrasonic diagnostic apparatus of the twentieth aspect, the image display device is supported by the arm (17) extending from the column support (19) along the processing device (4) or by the arm (17) extending from the processing device (4). Therefore, the space on the side of the operator with respect to the bed (13) is not occupied for supporting the image display device (3). As a result, the space for the operator becomes larger than the conventional space. Accordingly, the space in the room in which the ultrasonic diagnostic apparatus is installed can effectively be utilized.

In a twenty-first aspect of the invention, there is provided an ultrasonic diagnostic apparatus according to the twentieth aspect, wherein the arm (17) can rotate, expand or contract.

According to the ultrasonic diagnostic apparatus of the twenty-first aspect, the arm (17) can rotate or expand and contract. As a result, a spatial position or an orientation of the image display device can be changed.

In a twenty-second aspect of the invention, there is provided an ultrasonic diagnostic apparatus according to any one of the first to twenty-first aspects, wherein the operating device (1) and the processing device (4) have radio communication means (1a) and (4a), respectively, for mutual radio communication.

According to the ultrasonic diagnostic apparatus of the twenty-second aspect, a wire for connecting the operating device (1) with the processing device (4) can be eliminated. Therefore, the operating device (1) can be moved freely.

In a twenty-third aspect of the invention, there is provided an ultrasonic diagnostic apparatus according to any one of the first to twenty-second aspects, wherein the operating device (1) is mounted on a movable platform (18) with a caster.

According to the ultrasonic diagnostic apparatus of the twenty-third aspect, the position of the operating device (1) can freely be changed by moving the movable platform (18).

According to the ultrasonic diagnostic apparatus of the invention, it is possible to mount the processing device (4) on the mounting platform (5) or to raise the height of the processing device (4) without being influenced by the height of operating device (1). Therefore, it is possible to provide the cable receiving space (5a) inside the processing device (4) or the mounting platform (5). At the same time, it is possible to provide the opening (5b) in the cable receiving space (5a) at a high position from the floor (F). Accordingly, the cable (6) comes in and goes out at a position sufficiently higher than that of the patient lying on the bed (13), preventing the cable (6) from contacting and giving the patient discomfort.

The ultrasonic diagnostic apparatus of the present invention can be used as a stationary ultrasonic diagnostic apparatus.

Further objects and advantages of the present invention will be apparent from the following description of the preferred embodiments of the invention as illustrated in the accompanying drawings.

### BRIEF DESCRIPTION OF THE DRAWINGS

FIG. 1 is a partially exploded side view showing an ultrasonic diagnostic apparatus according to Embodiment 1.

FIG. 2 is a front view showing the ultrasonic diagnostic apparatus according to Embodiment 1.

FIG. 3 is a plan view showing the ultrasonic diagnostic apparatus according to Embodiment 1.

FIG. 4 is a partially-exploded enlarged side view of the vicinity of the opening.

FIG. 5 is a partially-exploded enlarged front view of the vicinity of the opening.

FIG. 6 is an enlarged side view of the vicinity of the movable pulley.

FIG. 7 is an enlarged side view of the vicinity of the movable-pulley lock (a state where the movable-pulley lock is released).

FIG. 8 is an enlarged side view of the vicinity of the movable-pulley lock (a state where the movable-pulley lock is working).

FIG. 9 is an enlarged side view showing a state where the cable lock is released.

FIG. 10 is a front view showing an ultrasonic diagnostic apparatus according to Embodiment 2.

### DETAILED DESCRIPTION OF THE INVENTION

Hereinbelow, preferred embodiments of the invention will be described with reference to drawings. The embodiments are not to limit the invention thereto.

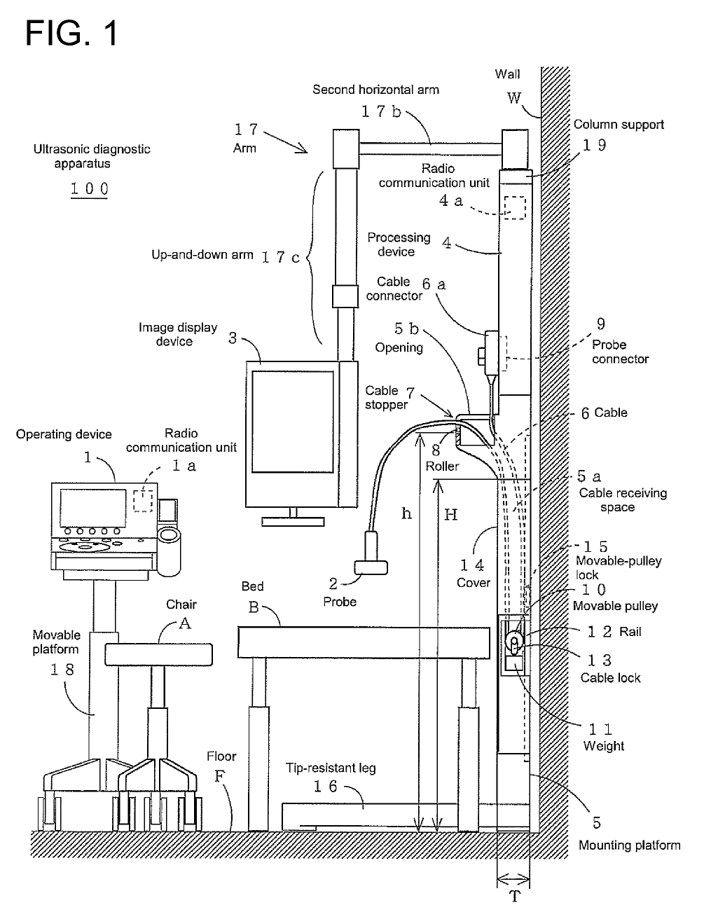

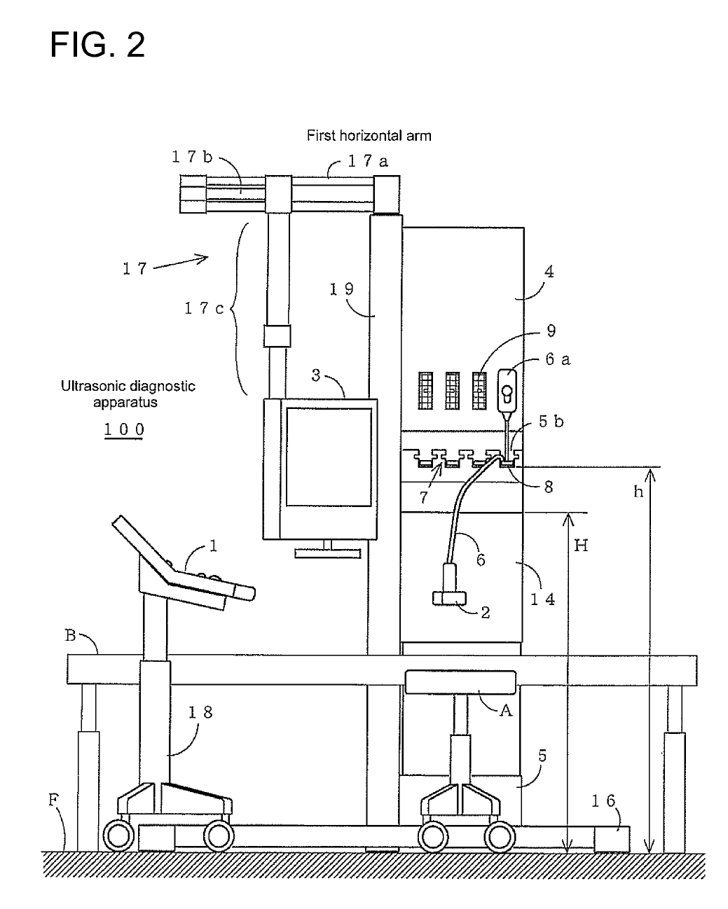

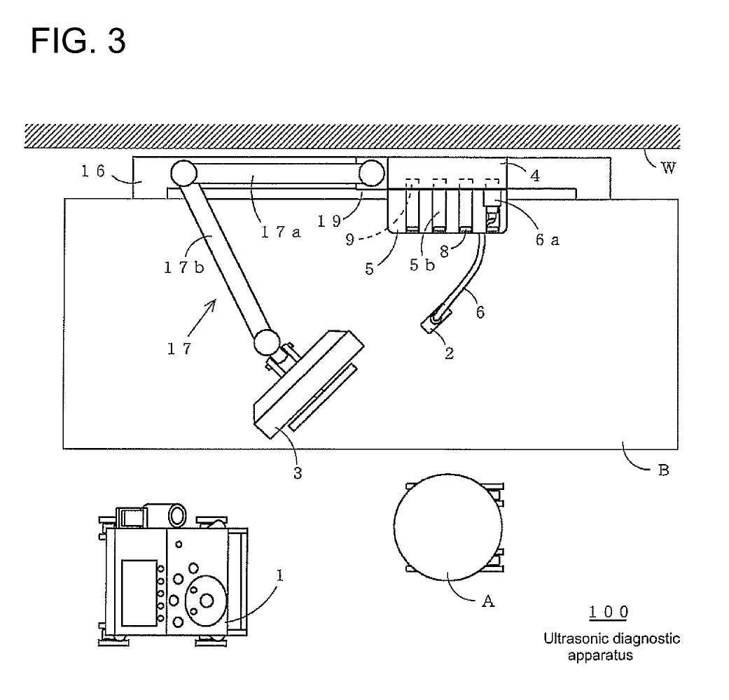

FIG. 1 is a partially exploded side view showing an ultrasonic diagnostic apparatus 100 according to Embodiment 1. FIG. 2 is a front view showing the ultrasonic diagnostic apparatus 100. FIG. 3 is a plan showing the ultrasonic diagnostic apparatus 100.

The ultrasonic diagnostic apparatus 100 basically includes: an operation device 1 for an operator to input an instruction; a probe 2 for transmitting and receiving ultrasonic waves; an image display device 3 for displaying an ultrasonic image; and a processing device 4 which controls driving the probe 1 based on the instruction by the operator, producing the ultrasonic image based on a received signal, and displaying it on the image display device 3.

The operation device 1 and the processing device 4 are formed separately. Further, the operation device 1 is installed on a movable platform 18 with a caster, and are placed on the side where a chair A on which the operator sits with respect to a bed B. Further, the operation device 1 has a radio communication unit 1a for radio communication with the processing device 4.

The processing device 4 is fixed on a mounting platform 5 placed between the bed B and a wall W. Further, the processing device 4 has a radio communication unit 4a for radio communication with the operating device 1 and a probe connector 9 for connecting a cable connector 6a of the probe 2.

Back surfaces of the processing device 4 and the mounting platform 5 are not uneven or their levels of unevenness are 1 cm or less, being substantially flat.

The height of the mounting platform 5 is 150 cm. The thickness of a portion of the mounting platform 5 up to a height H (100 cm) is T (10 cm). Further, a portion of the mounting platform 5 between 100 cm and 130 cm in height (h) is a projection, whose thickness is 20 cm.

An inner portion of the mounting platform 5 serves as a cable receiving space 5a for receiving the cable 6 of the probe 2. When a cover 14 of the mounting platform 5 is slid downwards, the inner portion of the mounting platform 5 between 65 cm and 100 cm in height (H) is exposed, allowing the operator to access the cable receiving space 5a.

Further, the mounting platform 5 has a tip-resistant leg 16. The tip-resistant leg 16 is preferably fastened to a floor F with anchor bolts.

The image display device 3 is supported by an arm 17 extending from a column support 19 fixed on side faces of the processing device 4 and the mounting platform 5.

The arm 17 includes: a first horizontal arm 17a extending horizontally from the column support 19; a second arm 17b extending horizontally from the first horizontal arm 17a; and an up-and-down arm 17c extending vertically from the second arm 17b.

The first horizontal arm 17a rotates horizontally at a joint with the column support 19. Further, the second horizontal arm 17b rotates horizontally at a joint with the first horizontal arm 17a.

The length of the up-and-down arm 17c is variable. Also, the up-and-down arm 17c rotates horizontally at a joint with the second horizontal arm 17b. The lower end of the up-and-down arm 17c serves as a joint that allows the image display device 3 to rotate within a vertical plane.

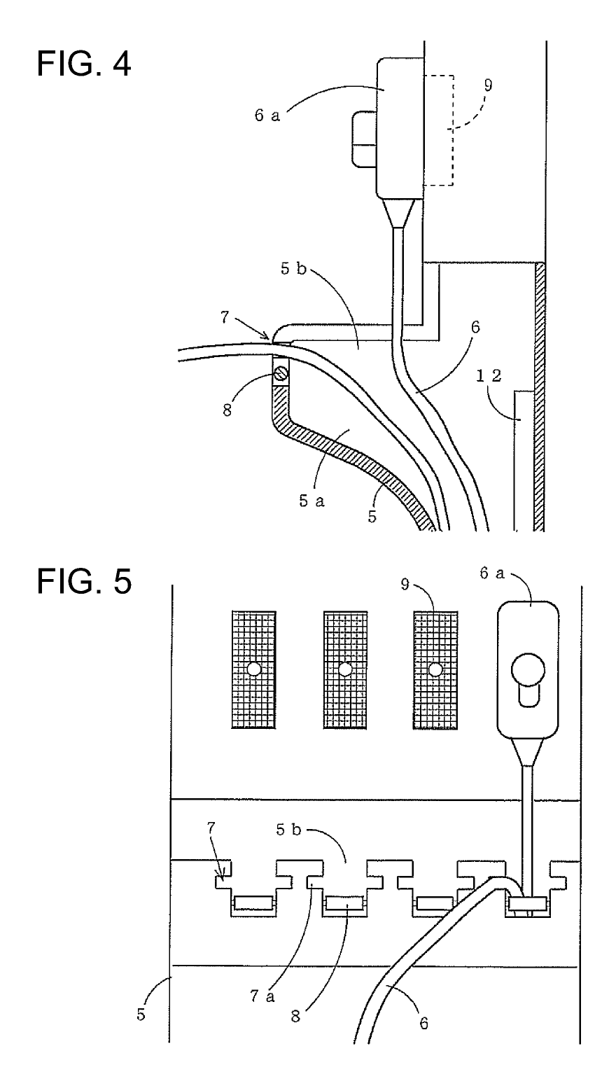

As shown in FIGS. 4 and 5, the projected portion of the mounting platform 5 between 120 cm and 130 cm in height serves as the opening 5b.

The cable 6 of the probe 2 is bent in the shape of U and is received in the cable receiving space 5a through the opening 5b.

A cable stopper 7 is formed in the opening 5b.

The cable stopper 7 is a slit 7a that is narrower than an outside diameter of the cable 6. By inserting the cable 6 in the slit 7a, the cable 6 is stopped in an engaged state by the elasticity of the cable 6.

Moreover, in the opening 5b, there is provided a roller 8 which may hit an undersurface of the cable 6.

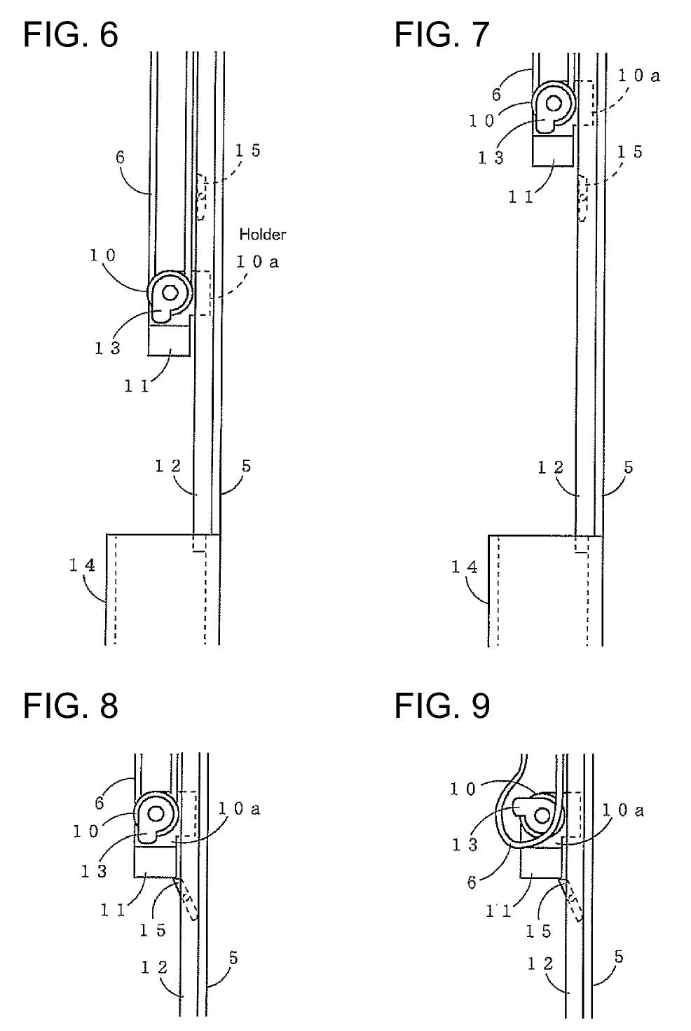

FIG. 6 shows a state in which the cover 14 is slid downwards and the inner portion of the mounting platform 5 between 65 cm and 100 cm in height (H) is exposed.

A movable pulley 10 whose diameter is 10 mm or larger, for example, is hung from a lower end of the U-shaped cable 6.

The movable pulley 10 is supported in a vertically movable manner, by a holder 10a, on a rail 12 fixed at right angles to the back surface of the mounting platform 5.

Also, the movable pulley 10 is pulled downwards by a weight 11.

A cable lock 13 is installed in the movable pulley 10.

The cable lock 13 includes a rotatable lever-like member.

A movable-pulley lock 15 is installed at a position of 70 cm in height of the rail 12 from the floor F.

The movable-pulley lock 15 is a seesaw-like member that moves in a seesaw-like manner and changes its position.

FIGS. 7 to 9 show steps to release the cable 6 from the movable pulley 10.

First, the cover 14 is slid downwards. In a state where the inner portion of the mounting platform 5 between 65 cm and 100 cm in height (H) is exposed, the cable 6 is pulled out. Further, as shown in FIG. 7, the movable pulley 10 is raised to a position above the movable-pulley lock 15.

Then, as shown in FIG. 8, the movable-pulley lock 15 is made to project, the movable pulley 10 is lowered, and the movable pulley 10 is mounted on the movable-pulley lock 15.

Next, as shown in FIG. 9, the cable lock 13 is rotated and raised by fingers to release the cable 6.

Steps to hang the movable pulley 10 on the cable 6 are carried out in a reversed manner.

According to the ultrasonic diagnostic apparatus 100 of Embodiment 1, the following effects are obtained.

(a) The operation device 1 and the processing device 4 are formed separately. Therefore, the processing device 4 can be mounted on the mounting platform 5, without being influenced by the height of the operation device 1. As a result, it is possible to provide the cable receiving space 5a inside the mounting platform 5 and to provide the opening 5b of the cable receiving space 5a at a position equal to 120 cm or higher from the floor F. Thus, the cable 6 comes in and goes out at a position sufficiently higher than that of the patient lying on the bed B, preventing the cable 6 from contacting and giving the patient discomfort.

(b) It is possible to receive the cable 6 of the probe 2 inside the mounting platform 5. Therefore, the cable 6 is prevented from being in the way.

(c) There is provided the cable stopper 7, which prevents the cable 6 from going out of the opening 5b or being pulled into the cable receiving space 5a against the operator's will.

(d) By inserting the cable 6 in the slit 7a, the cable 6 is stopped in an engaged state by the elasticity of the cable 6.

(e) The cable 6 can smoothly comes in and goes out of the opening 5b by the roller 8.

(f) The probe connector 9 is provided above the opening 5b. Therefore, the cable connector 6a of the cable 6 is attached to or detached from the probe connector 9 without being disturbed by the cable 6.

(g) The cable 6 is bent in the shape of U to be received into the cable receiving space 5a from the opening 5b. Accordingly, only one portion at the lower end of the U-shaped cable 6 is bent, preventing the cable 6 from being damaged.

(h) The movable pulley 10 prevents the cable 6 from bending with the curvature smaller than the radius (for example, 5 mm or over) of the movable pulley 10.

(i) The weight 11 attached to the movable pulley 10 provides a force to smoothly pulls the cable 6 into the cable receiving space 5a.

(j) Since the movable pulley 10 vertically moves while being supported on the rail 12, its stability and reliability is improved.

(k) Since the cable lock 13 is installed, the disconnection of the cable 6 from the movable pulley 10 is prevented, improving the stability and reliability.

(l) The cover 14 is vertically slid to allow the access to the inner portion of the mounting platform 5 below the position equal to or less than 120 em from the floor (F). As a result, it is easier to perform the maintenance work and becomes possible to remove the cable 6 of the probe 2 from the movable pulley 10 and to mount the cable 6 on the movable pulley 10.

(m) Since the movable-pulley lock 15 is installed, it is possible to hold the movable pulley 10 from which the cable 6 has been removed at the position equal to or higher than 60 cm from the floor, thereby making it easier to mount the cable 6 of the probe 2 on the movable pulley 10.

(n) The operating device 1 and the processing device 4 are formed separately. As a result, it is possible to place the operating device 1 on the side of the operator with respect to the bed B on which the patient is lying and to place the processing device 4 on the other side of the bed B. Further, if a spacing between the bed B and the wall W is 11 cm, for example, the processing device 4 can be installed along the wall W. As a result of the above, the space for the operator becomes larger than that of the conventional case, making it possible to effectively utilize the space of the room in which the ultrasonic diagnostic apparatus 100 is installed.

(o) The back surface of the processing device 4 or mounting platform 5 is substantially flat. Therefore, the processing device 4 can be installed along the wall W with almost no spacing provided.

(p) Although the length of the contact surface in the thickness direction of the mounting platform 5 is 10 cm or less, the mounting platform 5 can be prevented from tipping over by the tip-resistant leg 16.

(q) By moving the movable platform 18, the position of the operating device 1 can easily be changed.

(r) Since the wire for connecting the operating device 1 with the processing device 4 can be eliminated, the processing device 1 can freely be moved.

(s) The image display device 3 is supported by the arm 17 extending from the column support 19 provided along the processing device 4. As a result, the space on the side of the operator with respect to the bed B is not occupied for supporting the image display device 3. Thus, the space for the operator becomes larger than the conventional case, making it possible to effectively utilize the space of the room in which the ultrasonic diagnostic apparatus is installed.

(t) By rotating, expanding or contracting the arm 17, the spatial position and orientation of the image display device 3 can be changed.

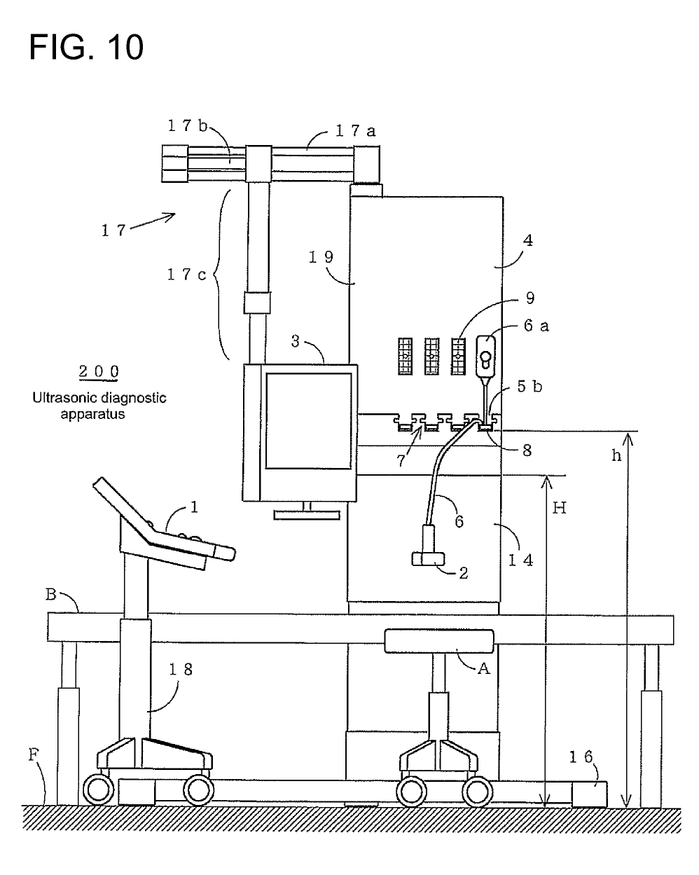

FIG. 10 is a front view showing an ultrasonic diagnostic apparatus 200 according to Embodiment 2. In the ultrasonic diagnostic apparatus 200, the mounting platform 5 and the column support 19 of Embodiment 1 is integrally formed with the processing device 4.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus, comprising:
an operating device for an operator to input an instruction;
a probe configured to transmit and receive ultrasonic waves;
an image display device configured to display an ultrasonic image; and
an processing device configured to drive said probe based on the input instruction, produce the ultrasonic image based on a received signal, and to display the ultrasonic image on said image display device, wherein said processing device is one of mounted on an installation platform and placed on a floor, a cable receiving space is provided inside one of said processing device and said installation platform and is configured to receive a cable of said probe, a probe connector is provided above an opening of said cable receiving space such that said cable is received into said cable receiving space in the shape of a U from said opening, a movable pulley is hung from a lower end of said U-shaped cable, and said movable pulley is supported on a rail in a vertically movable manner.

2. An ultrasonic diagnostic apparatus, comprising:
an operating device for an operator to input an instruction;
a probe configured to transmit and receive ultrasonic waves;
an image display device configured to display an ultrasonic image; and
an processing device configured to drive said probe based on the input instruction, produce the ultrasonic image based on a received signal, and display the ultrasonic image on said image display device, wherein said operating device is formed separately from said processing device, said processing device is one of mounted on an installation platform and placed on a floor, a cable receiving space is provided inside one of said processing device and said installation platform for receiving a cable, an opening is provided at a position equal to or higher than 120 cm from the floor, and said cable is received in said cable receiving space through said opening.

3. An ultrasonic diagnostic apparatus according to claim 2, wherein a probe connector is provided above said opening of said processing device.

4. An ultrasonic diagnostic apparatus according to claim 3, wherein said cable is bent in a U-shaped manner from said opening and is received in said cable receiving space.

5. An ultrasonic diagnostic apparatus according to claim 4, wherein a movable pulley is hung from a lower end of said U-shaped cable.

6. An ultrasonic diagnostic apparatus according to claim 5, wherein said movable pulley is supported on a rail in a vertically movable manner.

7. An ultrasonic diagnostic apparatus according to claim 1, wherein said movable pulley is urged downwards by a weight.

8. An ultrasonic diagnostic apparatus according to claim 1, wherein a cable lock is provided to prevent the disconnection of said cable from said movable pulley.

9. An ultrasonic diagnostic apparatus according to claim 8, wherein said cable lock is comprised of a rotatable lever-like member.

10. An ultrasonic diagnostic apparatus according to claim 8, wherein a cover is provided over a portion of one of said processing device and said mounting platform at approximately 120 cm or less from the floor, said cover is configured to slide vertically to allow access inside the one of said processing device and said mounting platform.

11. An ultrasonic diagnostic apparatus according to claim 10, wherein said cable is attached to or detached from said movable pulley when said cable lock is released.

12. An ultrasonic diagnostic apparatus according to claim 10, wherein a movable-pulley lock is provided for holding said movable pulley at a position of said rail equal to or higher than 60 cm from the floor.

13. An ultrasonic diagnostic apparatus according to claim 12, wherein said movable-pulley lock is comprised of a seesaw-like member that varies its position in a seesaw-like manner.

14. An ultrasonic diagnostic apparatus according to claim 1, wherein a cable stopper is provided for preventing said cable from coming into and going out of said cable receiving space.

15. An ultrasonic diagnostic apparatus according to claim 14, wherein said cable stopper comprises a slit that is narrower than an outside diameter of said cable.

16. An ultrasonic diagnostic apparatus according to claim 1, wherein, in said opening, a roller is provided that may hit an undersurface of said cable.

17. An ultrasonic diagnostic apparatus according to claim 1, wherein a thickness of one of a portion of said processing device and said mounting platform at a position equal to or lower than 70 cm from the floor, is 10 cm or less.

18. An ultrasonic diagnostic apparatus according to claim 1, wherein one of said processing device and said mounting platform comprises a tip-resistant leg.

19. An ultrasonic diagnostic apparatus according to claim 1, wherein said image display device is supported by one of an arm extending from a column support provided along said processing device and an arm extending from said processing device.

20. An ultrasonic diagnostic apparatus according to claim 1, wherein said operating device and said processing device each comprises radio communication means for mutual radio communication.
